(19) United States
(12) Patent Application Publication
(10) Pub. No.: US 2010/0241018 A1
(43) Pub. Date: Sep. 23, 2010

(54) BABY MONITOR

(76) Inventor: PETER SAMUEL VOGEL, SYDNEY (AU)

Correspondence Address: PETER VOGEL, 30 ADELINE STREET, FAULCONBRIDGE, 2776, AU

(21) Appl. No.: 12/724,434

(22) Filed: Mar. 16, 2010

(30) Foreign Application Priority Data
Mar. 21, 2009 (AU) 2009901220

(51) Int. Cl. A61B 5/04

(52) U.S. Cl. 600/511

(57) ABSTRACT

This baby monitor provides monitoring of a baby's activities and vital signs from a remote location, possibly anywhere in the world. A sensor unit placed near the baby receives input from the baby, typically sound, image and vital signs such as heartbeat, respiration and temperature. Environmental conditions such as ambient temperature can also be monitored. One embodiment of the invention is worn on, or close to, a baby's chest while sleeping and monitors heartbeats and generates an alarm if the heart rate drops unexpectedly, indicating distress such as lack of oxygen. The invention can also be used for parental reassurance by indicating the baby's heartbeat through a pulsating LED or audible sound.

### FIELD OF THE INVENTION

The present invention relates to systems for monitoring an infant's activities and vital signs, in particular while sleeping.

### BACKGROUND OF THE INVENTION

Although the incidence of SIDS (sudden infant death syndrome) has halved over the last decade, it still claims 1 in 2000 babies in their first 6 months of life. Between 1985 and 2005, deaths from SIDS in Australia declined by 83%, from 523 deaths in 1985 to 87 in 2005, thanks largely to promotion of safer practices (such as placing the baby to sleep on their back).

Despite this decline, SIDS is still a significant concern. Importantly, death can easily be averted if the carer is aware that the baby stops breathing. Gentle stimulation will usually cause the baby to restart breathing and if the baby has only stopped breathing for a minute or so it will be unharmed.

It should also be noted that SIDS is a diagnosis of exclusion, that is the term is only applied if there is no known cause of death. If the cause of death is discovered, such as suffocation or apnoea, it is not recorded as SIDS. So the rate of infant death while sleeping is much higher than the SIDS figures.

Various baby monitors have been on the market for decades. These typically provide remote monitoring of the baby's sounds, and in some cases an image of the baby is made available as well.

However, when the baby is asleep, it may be making no discernable sound or movement. Parents using such monitors are sometimes unsure that their baby is alive and well when it appears to be sleeping soundly.

Some alarms are designed specifically for monitoring sleeping babies. The type commonly used at home monitor movements of the baby as an indication of breathing. It is common for babies to breathe very lightly, often moving just their diaphragm which these devices can't detect, resulting in false positive alarms which frighten parents and disturb baby's sleep. These frequent false alarms make these devices difficult to live with and parents often abandon their use.

There are also many reports of false negatives, for example when the baby has been removed from the cot and the alarm does not sound. Due to the extreme sensitivity required to detect the movement of a baby breathing lightly, even air currents caused by wind or fans can falsely be registered as breathing. Similarly, if a baby is choking or fitting the alarm will not sound as movement is still being produced.

In a hospital setting, babies can be connected to machines which monitor heart rate and oxygen saturation as well as breathing. This is a more effective system, however these machines are expensive and require sensors attached to the baby, making them impractical for long term home use.

### SUMMARY OF THE INVENTION

It is an object of this invention to provide a baby monitor which offers caregivers the reassurance that their baby is alive and well, even when it is sleeping silently.

It is another object of this invention to provide an infant distress alarm which is safe, convenient, affordable and which does not suffer the high rate of false positive or false negative alarms of prior art.

It is yet another object of the invention to provide a vital signs monitor which provides reassurance that an infant's vital signs are normal.

It is another object of the invention to provide a baby monitor which allows caregivers to monitor their baby from a great distance.

It is yet another object to provide a baby monitor which allows caregivers to monitor their baby's environment.

It is yet another object to provide a baby monitor which monitors a baby's orientation and/or movement.

In one aspect, the present invention provides a baby monitor which detects at least one of a baby's vital signs and allows remote access for monitoring these vital signs from anywhere in the world where a suitable communications medium is available. Appropriate vital signs include sound, vision, movement, heartbeat, respiration, body temperature, or any other information generated by the baby.

In some embodiments, the invention uses the Internet to provide access to the monitored vital signs or environmental data.

In other embodiments, the invention uses a telephone network to provide access to the monitored vital signs or environmental data.

In other embodiments, the invention is further adapted to generate alarms when certain conditions are met. For example, the invention can remotely alert parents if the baby's heart rate drops by a predetermined proportion of a measured baseline within a predetermined time period, if the ambient temperature becomes too hot or too cold, if the baby cries for longer than a predetermined time, if the baby's body temperature is out of bounds, or if the baby is absent from the monitored area for too long. The invention can also convey position information, for example where the baby is within the home, or whether the baby is upright, prone, or prostrate.

In some embodiments, the invention monitors the baby's orientation and/or movement and optionally provides an alarm if certain orientation and/or movement conditions are met, for example if the baby is not in the desired position, such as supine when sleeping.

The invention can be further adapted to provide trend data, for example by displaying on a website the baby's heartrate, temperature, position, movement, crying over a period of time. Monitored information can also be stored short or long term.

In another aspect, the present invention provides an infant distress alarm comprising a wearable enclosure, heart rate measuring means and alarm means adapted to indicate when heart rate deviates outside prescribed bounds. In some embodiments the wearable enclosure is worn in contact with the infant's body. In other embodiments the wearable enclosure is separated from the infant's body by one or more layers of clothing. For example, the enclosure of the invention can be attached to the outside of a vest or pyjama.

In some embodiments, the invention is adapted to alarm when heart rate drops below a predetermined absolute rate for a predetermined time. For example, it is known that a heartbeat below 60 BPM and sustained for more than 10 seconds is probably a sign of distress in an infant.

In other embodiments, the invention is adapted to alarm when heart rate drops by a predetermined proportion of a measured baseline within a predetermined time period. For example, it is known that a drop of heartbeat below 70% of baseline in less than 60 seconds and sustained for more than 10 seconds is probably a sign of distress in an infant.

In other embodiments, the invention is adapted to alarm when heart rate becomes too fast or increases suddenly.

According to another aspect of the invention, further processing means are provided to detect other bodily actions, for example moving, fitting, shivering, breathing, coughing, apnea, choking. In some embodiments these detected actions can be made to trigger an alarm if certain bounds are exceeded. In other embodiments, detected actions can be used to qualify other alarms. For example, movement detection can be used to suppress an alarm which would otherwise be triggered by loss of heartbeat sensing.

In some embodiments the heart rate measuring means and alarm means are located within the same wearable enclosure, in other embodiments wireless communication is provided so that the alarm means can be remote from the heart rate measuring means, for example in another room of the house.

In another aspect, the invention provides a parental reassurance device comprising a wearable enclosure, heart beat detection means, and signalling means coupled to said heart beat detection means and adapted to reproduce a periodic signal at the rate of the heart beat. In some embodiments the reproduced signal is a sound, in other embodiments the signal is visual, and in yet other embodiments it is a tactile signal. In this aspect the invention can allow a parent to hear, see or feel the rhythm of a baby's heartbeat without coming into contact with the baby.

In some embodiments the signalling is a sound reproduced by processing heartbeat sounds in real time. In other embodiments the signalling is a synthesised sound of the same period as the detected heart beat but not necessarily in phase with the detected heart. For example, the invention can detect a heartbeat and produce a tick, beep, thump or other desirable sound after a short delay, typically 250 ms. This aspect of the invention can be advantageous in avoiding feedback problems between the detecting device and the sound generating device.

In some embodiments the signalling means can be located within the same wearable enclosure as the heart beat detection means, in other embodiments wireless communication can be provided so that the signalling means can be remote, for example in another room of the house.

Wireless communication of this invention can utilize any of the technologies well known to the art, such as radio frequency, magnetic, optical, acoustic, ultrasonic signaling. In some embodiments, where only a short range required, near field or personal area networking techniques can be employed with good results.

The heart rate measuring means of the invention can comprise a movement sensor, electric activity detector, electrical resistance detector, optical sensor, acoustic transducer, or any other device capable of detecting heartbeats. According to one preferred embodiment, the heart rate measuring means comprises an accelerometer, for example a MEMS device. In another preferred embodiment, the heart rate measuring means comprises a piezoelectric vibration transducer. In another embodiment the heart rate measuring means comprises a microphone, preferably optimized for a frequency bandwidth of 20-40 Hz. In yet another embodiment, the heart rate measuring means comprises a piezo fibre composite material which generates electric output as it is flexed. In another embodiment, the heart rate measuring means comprises a light emitter and detector adapted to measure transmission of light through skin and detect changes in transmission due to pulsing of the blood.

According to one extension of the invention, means is also provided for logging data. In one such preferred embodiment solid state memory is provided and the processing means of the invention is adapted to store in this memory data such as heart rate and alarms, along with timing information so that data can be retrospectively analysed. The timing information can be time/date data. In one preferred embodiment, the timing information is a sequential count of seconds which need not be set to a particular time/date reference, and the time of a particular data recording is calculated by reading the latest count at the time of data retrieval, and calculating backwards from the time/date at which the data was retrieved. This embodiment has the advantage that even though it is not necessary to set or maintain the time/date within the invention, it is possible to accurately know the time/date of any part of a recording.

In some embodiments of the invention, stored data includes heart rate data. In other embodiments, stored data includes heart beat information. Heart beat information can be raw data detected by the heart beat transducer, or it can be signals produced by processing the raw data, for example after bandpass amplification or filtering.

According to another extension of the invention, means is also provided for communicating data from the invention to another system. In some embodiments an interface is provided for connection to a computer. In other embodiments the logging memory of the invention is removable and can be connected to another device, such as a computer, for transferring stored data. In yet other embodiments a wireless communication interface is provided so that the invention can transmit data, including heart rate information or alarms, to other computing systems, for example via the Internet.

According to another extension of the invention, means is also provided for measuring other physiological parameters such as body temperature, indicating or communicating this information, or generating an alarm if such a parameter exceeds preset bounds.

### DESCRIPTION OF PREFERRED EMBODIMENTS

Embodiments of the invention will now be described with reference to the drawings in which:

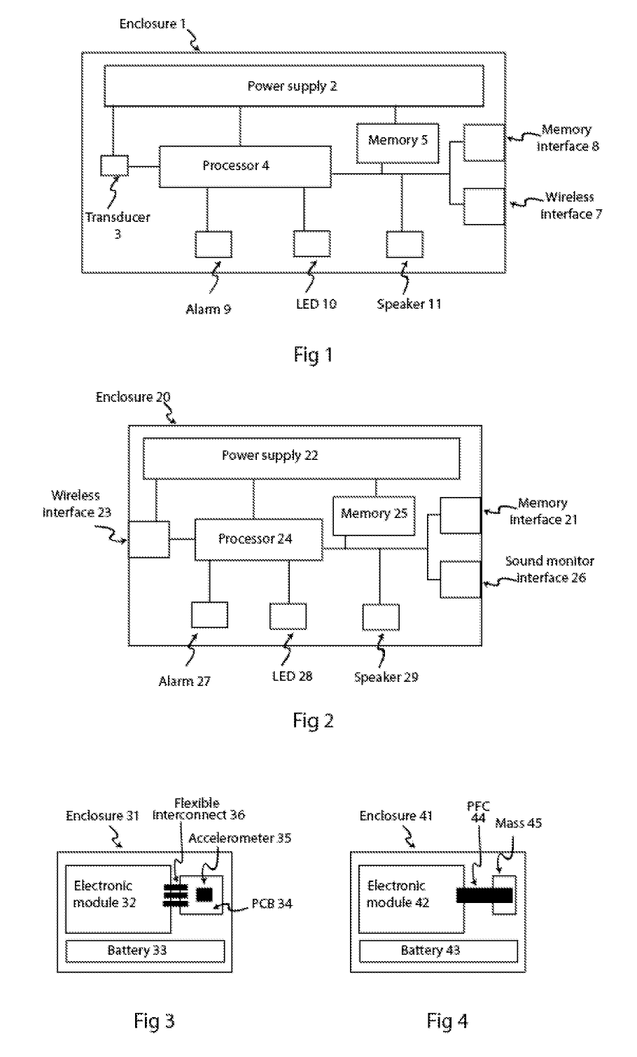

FIG. 1 is a block diagram of an embodiment of the present invention which can be used as a stand-alone device;

FIG. 2 is a block diagram of a base station which can be used with the system of FIG. 1 to provide remote monitoring;

FIG. 3 is a block diagram of an embodiment of the present invention in which the transducer is an accelerometer;

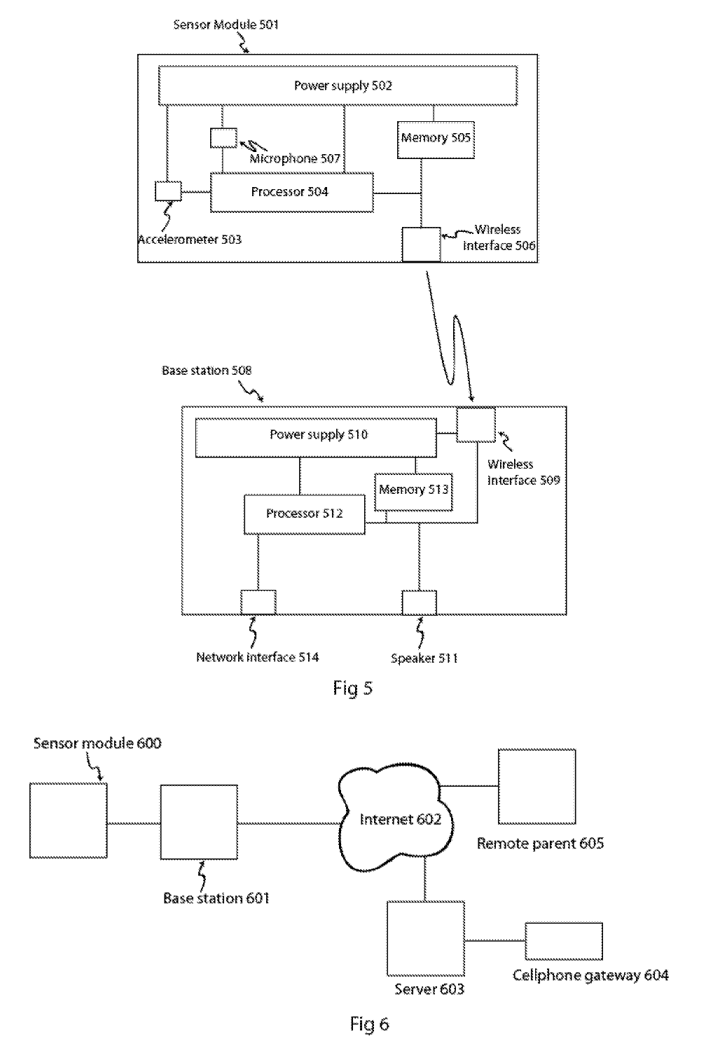

FIG. 4 is a block diagram of an embodiment of the present invention in which a piezo-fibre composite material is the transducer.

FIG. 5 is a block diagram of an embodiment of the present invention which monitors a baby's sounds and heartbeat and provides remote access via telephone or internet; and

FIG. 6 is a block diagram of an embodiment of the invention utilising a centralised server as the remote access mechanism.

Referring now to FIG. 1, an embodiment of the invention is shown in which the sensing and alarm elements of the invention are included in the same enclosure. This embodiment of the invention can operate as a stand-alone device. Enclosure 1 is a watertight plastic case, approximately 30 mm×50 mm×5 mm which attaches to the baby's clothing using a pocket, adhesive, clips, Velcro or other suitable means. It can also be attached by means of an elastic strap or belt, or be integrated into an item of clothing. For convenience a fabric pocket can be provided which is fixed to the clothing, for example by clips or iron-on adhesive, and the invention slips into the pocket when in use and can easily be removed for washing the clothes or recharging the invention.

Power supply 2 in this embodiment is a rechargeable battery and powers all components within enclosure 1.

Transducer 3 in this embodiment is a MEMS accelerometer, oriented so that the axis of maximum sensitivity is perpendicular to the large surface of enclosure 1. Processor 4 includes a band pass amplifier which amplifies the signal from transducer 3 in approximately the band 20-40 Hz. The output of this amplifier feeds an ADC input of a low-powered microcontroller within processor 4. The microcontroller samples the ADC input at 250 Hz and scales the signal to a suitable amplitude.

Speaker 11 provides a heartbeat sound for the “reassurance” function of the invention. However the selected heartbeat waveform has a spectral peak around 25 Hz, a frequency which is difficult for human hearing and which small loudspeakers cannot effectively reproduce. In order to make a more audible sound, the microcontroller of processor 4 ring modulates a higher frequency carrier, say 400 Hz, with the filtered output of transducer 3 before feeding speaker 11. Speaker 11 therefore needs only to have a frequency response down to 400 Hz, which is inexpensively achievable in a small package such as a piezo device.

In another variation of this embodiment of the invention, the reassurance sound is not reproduced directly from the heartbeat detected by transducer 3 but is a synthesised sound triggered by detected heartbeats. In that case the reassurance sound can be any sound desired, for example a beep or the chirping of a cricket. The invention can even produce music which is locked in synch with the detected heartbeat, which may have a calming effect.

Depending on the device used as transducer 3, feedback from speaker 11 could compromise heartbeat detection. To ameliorate this problem the processor 4 can be adapted to produce the reassurance sound delayed from the detected heartbeat.

Processor 4 also drives LED 10 which is a visual indicator flashing for about 150 ms each heart beat.

Processor 4 also detects the frequency of the received heartbeats, calculating the rate in beats per minute or the period in milliseconds. Software is provided for generating an alarm from Alarm 9 if the calculated heart rate meets certain tests.

Alarm 9 can be a signalling device such as a piezo buzzer, or the invention can use the same device as speaker 11 for the alarm sound. The test for an alarm condition in this embodiment is:

a) Heart rate less than 60 b.p.m. for more than 10 seconds, or

b) Heart rate drops more than 30% in less than 60 seconds

This algorithm uses constants stored in non-volatile memory within processor 4 so that the values can easily be adjusted as circumstances require.

If an alarm condition is detected, LED 10 can optionally be made to flash in a distinctive pattern as well.

When an alarm condition is detected, the alarm output is activated for a predetermined minimum time.

To minimise the incidence of false alarms, processor 4 is adapted to differentiate between low heart rate and noise signals caused, for example, by the baby's movements, coughing, hiccups etc. In the simplest case, random movements which swamp heart rate measurements can be assumed to be a sign that the baby is healthy and the alarm is inhibited. Suitable processing can also be provided to differentiate between baby's normal movements and fits.

Memory 5 is a non-volatile memory device, such as FLASH or battery powered SRAM, which stores a historical recording of heartbeat data. Data can be stored in raw form, which is useful for later detailed analysis of waveforms etc. or in compressed form such as heart rate or period. The latter uses far less memory than the former, so much longer history can be stored in a given amount of memory. In a preferred embodiment, 64 MB of memory is provided, which is sufficient to store more than 24 hours of raw data or many months of compressed data.

In some embodiments, timestamps are stored along with the data in memory 5. These can be absolute or relative timestamps. Absolute timestamps convey the time and date of the recorded data and require processor 4 to include a non-volatile time/date clock which needs to be set to the correct time/date before the invention is used. Relative timestamps use a counter which generates a sequential count of time periods, typically one count per second. The value of the counter is used as the timestamp stored along with recorded data. To determine the actual time of a particular recording stored in memory 5, the value of the counter is read at the time of reading the memory back. The reading device can then calculate absolute times using the present counter value as a reference to the present time.

Wireless interface 7 can optionally be used to send information from the body-borne unit to another device, for example a wireless network connected to the internet, a personal computer, or a base station designed to reproduce the outputs of the invention at a location remote from the child being monitored, for example in another room of the house. To conserve battery power and to ensure that the baby is not exposed to any hazardous electromagnetic radiation, wireless interface 7 employs a communications technology which is very low power. In this exemplary embodiment, a micropower 13.56 MHz radio link is used to communicate to a base station located within 2 meters of the child being monitored. The use of a low frequency is preferred to microwave frequencies as used by Bluetooth or WiFi as absorption by the human body is much lower at greater wavelengths. For even lower power operation, the invention can utilise passive near-field communication techniques, that is the body-worn device transmits data to the base station by load modulation.

In another variation of this preferred embodiment, wireless interface 7 uses infra-red communication to a nearby base station. This has the advantage of not generating any radio waves and is highly immune to interference. One disadvantage is that the path between transmitter and receiver must be unobstructed. If the baby rolls over onto its stomach, for example, signal will be lost. This is not a problem in applications where the invention is used to monitor a young baby since supine sleeping reduces risk of SIDS. Although infra-red LEDs require quite high drive currents, the duty cycle can be greatly restricted to obtain the required low power consumption.

Referring now to FIG. 2, a base station suitable for use with the device of FIG. 1 will be described. The embodiment of the invention of FIG. 1 is capable of practising the invention as a stand-alone device, which is a useful embodiment in many situations, for example when a carer is in the same room as the monitored baby, or where a conventional sound monitor is located near the baby, in which case an alarm sound would be conveyed to the carer in the same manner as the baby's cry. However in some cases it is desirable to extend the function of the invention to a point remote from the monitored baby. In such circumstances the invention comprising the apparatus of FIG. 1 and the base station of FIG. 2 together form an embodiment of the invention in which the functionality is split between the body-worn device and a remote base station.

In this example, enclosure 20 is located a few meters from the baby being monitored by the apparatus of FIG. 1. Wireless interface 7 of FIG. 1 transmits heartbeat data which is received by wireless interface 23 of FIG. 2. Processor 24, memory 25, memory interface 21, alarm 27, LED 28, and speaker 29 function as described in relation to the corresponding components of FIG. 1. Power supply 22 in this embodiment is preferably adapted to receive mains power as an alternative to, or in addition to, battery. Sound monitor interface 26 optionally provides a connection to a conventional “baby monitor” which transmits the sounds of the baby to a carer located elsewhere in the house. The signal which feeds speaker 29 and the alarm signal that feeds alarm 27 are output to sound monitor interface 26 so that an electrical connection can be made to the input of the separate baby monitor. Alternatively, the system of FIG. 2 can simply be placed near the baby monitor and the sound transmitted acoustically to the baby monitor and hence the carer. An advantage of electrical connection is that a control can be provided to allow the carer to adjust the level of reassurance sound and alarm sound relative to the usual acoustic transmission. In another version of the invention the base station of FIG. 2 is incorporated into the same device as the acoustic baby monitor.

If the invention is used with a base station as described above, the device worn by the baby (FIG. 1) can be simplified if desired, for example by removing all but transducer 3, processor 4, power supply 2 and wireless interface 7.

The wireless interface of FIG. 1 or FIG. 2 can also be used to allow data to be communicated to another computing device, for example a home PC or a remote server via the Internet. One extension of the invention provides logging of baby's sleep patterns over time, optionally including biometric data as well. This logging can be performed by the home PC or a remote service. Other information about the baby's health can be entered into the logging system to provide a useful profile of the baby's health and development. For example, parents can use a website to enter details of the baby's meals and that can be correlated to sleep data to identify possible food allergies.

Referring now to FIG. 3, an embodiment of the invention is shown in which the heart beat transducer is a MEMS accelerometer. In this example the accelerometer is optionally attached to main electronic module 32 of the unit by flexible interconnect 36 so that accelerometer 35 is free to vibrate independently of the PCB. This can be advantageous as the mass of the PCB or other components, especially the battery, can absorb vibration the device is trying to detect as the subject's heart beats. Further improvement of signal detection can be attained by arranging the mechanical characteristics of flexible interconnect 36, accelerometer 35 and PCB 34 so that the assembly is mechanically resonant at an optimal frequency, for example 25 Hz.

FIG. 4 shows the arrangement of components in an embodiment of the invention using piezo-fibre composite (PFC) material as the transducer. In this example PFC 44 is attached to an extremity of electronic module 42 and a suitable mass 45 is attached to the end of the PFC so that vibration maximizes flexing of PFC 44. Again it may be desirable to tune the PFC and the mass so the assembly is mechanically resonant at a frequency of interest, say 25 Hz.

Referring now to FIG. 5, sensor module 501 is a lightweight enclosure which is worn by the baby. Accelerometer 503 detects the baby's heartbeat, and microphone 507 detects the baby's sounds. Processor 504 receives these signals and after suitable processing sends them to wireless interface 506. Memory 505 is used by processor 504 as working memory and power supply 502, conveniently a rechargeable battery and suitable regulators, provides power for all components. Base station 508 is a unit which is located some distance, for example within 20 meters, from the baby and sensor module 501, the distance being limited by the range of the wireless interface. The wireless signals from wireless interface 506 are received by wireless interface 509 and processed by processor 512, which uses memory 513 as working memory. Sound signals originating from microphone 507 are recovered and fed via a suitable amplifier to speaker 511. This allows the person near the base station, typically located several meters away from the monitored baby, to hear sounds made by the baby. Heartbeats detected by accelerometer 503 are also decoded by processor 512, and after suitable processing, to produce a readably audible facsimile of a heartbeat, these sounds are also fed to speaker 511. Volume control means are also typically provided to allow the user to adjust the volume of the baby's sounds and its heartbeat sounds independently.

Network interface 514 allows access to the data received by processor 512 via a suitable external network. This network can for convenience be the internet, in which case network interface 514 is an ethernet interface or a wireless interface such as WiFi. In some embodiments, network interface 514 is an interface to a telephone network, such as a cellular wireless interface or a wired telephone line.

In some embodiments, processor 512 is adapted to provide a web server which can be accessed via the internet so that the baby's vital information can be retrieved over the internet using a standard web browser. In other embodiments, processor 512 is adapted to deliver the baby's vital data to a remote telephone caller. Data can be delivered in audio form, text form, graphical form or any other convenient form appropriate to the communications medium used for remote access. When remote telephone access is used, an interactive voice prompt system can be used to prompt the caller to enter a digit identifying what data they are seeking. For example, “Dial 1 to hear your baby's sounds, 2 to hear heartbeat, 3 for room temperature, 4 to speak to the carer”. If 3 is entered, for example, a voice synthesiser can speak the room temperature. If 4 is entered, processor 512 connects the call to the speaker and relays your voice to the person near the base unit. The invention can be further adapted to provide means for the carer to talk back, or for the caller's voice to be heard by the baby.

Whereas this embodiment describes the invention as providing access by remote login via the internet or phone, the invention can also be practised with good results using the invention as the communication originating device. For example, the invention can be adapted to initiate a call or SMS message to a remote parent on detecting certain conditions. Messages such as “baby is asleep” or “baby is crying” can be spontaneously sent via SMS. Similarly, messages can be sent by email or live chat messaging.

Referring now to FIG. 6, an embodiment of the invention utilising a centralised server as the remote access mechanism will be described. Whereas the embodiment described in FIG. 5 includes a web server for remote browser access, in this embodiment a server located outside the base station can be used. For example, a centralised server can communicate with one or many base stations via the Internet.

In this example, base station 601 receives data from sensor module 600 as described in relation to FIG. 5 above, and communicates the baby's data to server 603 via internet 602. Remote telephone access is provided at the server by cellphone gateway 604, and remote access for parent 605 is provided via internet 602. In this manner, cost and complexity of the invention can be reduced by sharing one facility across many users. Server 603 can also perform much more complex functions than would be practical to provide in individual home base stations. For example, server 603 can include a database which stores information about individual monitored babies over time, so that, for example, parents can view historical data about their baby's status. This can also be useful for medical or other research purposes, for example by accumulating large population samples of babies' heartbeats over long periods. Large longitudinal studies of factors preceding sudden infant death or other health issues could thereby be readily conducted.

It will be understood that while certain preferred embodiments of the invention are described above, many variations can be made without departing from the scope of the invention.

For example, where the invention is described as being an infant distress alarm, it is also applicable to older people or animals. Furthermore, it need not be used as a distress alarm; it can be used purely as a reassurance device, novelty device or any other purpose.

Whereas the invention is described as being attached to clothing over the chest, it can also be attached by different means, for example by adhesion to the skin, worn around the wrist or ankle, held to the forehead by a headband or hat, and so on. It is also not essential that the invention be attached to the child or its clothing, it can simply be in contact with the child or clothing. For example, the invention can be placed on the chest of a sleeping baby and it will stay in position until the baby moves. It can also be placed underneath the sleeping baby.

Although the invention has been found to function well using an accelerometer as the transducer, many other types of transducer can be used with good effect. For example a large variety of microphones are also suitable. Other forms of heart rate detection can also be used, such as ECG or optical pulse detection.

The invention can also be used as a measuring, logging or monitoring device, without an alarm.

Whereas the exemplary embodiments of the invention described herein use heartbeat as a vital sign, the invention can also be used to monitor other vital signs such as body temperature, breathing and so on. Breathing can be detected using an accelerometer, strain gauge or other well-known technique. Furthermore, one transducer can be used for multiple purposes, for example detecting heartbeat and baby's voice.

Whereas the exemplary embodiments describe certain algorithms for generating alarm conditions, other algorithms can be utilised. For example, it may be desirable to detect tachycardia, arrhythmia, or other irregularities. The invention can also be adapted to detect waking or sleeping, breathing, movement, coughing, choking, fitting and so on. In some embodiments multiple detected vital signs can be used as inputs to the alarm algorithm.

It will also be understood that while the invention is described herein as being used in a domestic setting, it is also very useful in a clinical or hospital setting. In such cases it may be desirable to utilize many of the inventive devices with a single room, in which case the devices can be adapted to include a unique identifying number and wireless networking techniques can be applied to allow many devices to operate simultaneously without interference. In some embodiments multiple child-worn devices can communicate with a single base station, facilitating monitoring of several babies from the one point. The invention can be similarly adapted for use in a home where more than one baby lives.

Whereas the exemplary embodiments of the invention described herein use heartbeat as a vital sign, the invention can also be used to monitor other vital signs such as body temperature, breathing and so on. Breathing can be detected using an accelerometer, strain gauge or other well-known technique.

Whereas the exemplary embodiments describe certain algorithms for generating alarm conditions, other algorithms can be utilised. For example, it may be desirable to detect tachycardia, arrhythmia, or other irregularities. The invention can also be adapted to detect waking or sleeping, breathing, movement, coughing, choking, fitting and so on. In some embodiments multiple detected vital signs can be used as inputs to the alarm algorithm.

Various methods of powering the invention are also envisaged, such as rechargeable or non-rechargeable battery. The invention can also be passively powered, taking the required energy from an electric or magnetic field. In embodiments using a piezoelectric transducer, the transducer can also be used to generate power required by the invention, or to recharge the battery, when the device is moved vigorously.

1. A baby monitor comprising means for sensing a baby's vital signs and means for accessing said sensed vital signs from a remote location.

2. A baby monitor as claimed in claim 1, wherein the means for sensing a baby's vital signs is a heartbeat detector.

3. A baby monitor as claimed in claim 1, wherein the means for sensing a baby's vital signs is a microphone.

4. A baby monitor as claimed in claim 1, wherein the means for accessing said sensed vital signs from a remote location is the internet.

5. A baby monitor as claimed in claim 1, wherein the means for accessing said sensed vital signs from a remote location is a telephone.

6. A baby monitor comprising a wearable enclosure, heart rate measuring means and alarm means adapted to indicate when heart rate deviates outside prescribed bounds.

7. A baby monitor according to claim 6 in which the wearable enclosure is worn in contact with the infant's body.

8. A baby monitor according to claim 6 in which the wearable enclosure is separated from the infant's body by one or more layers of clothing.

9. A baby monitor according to claim 6 adapted to alarm when heart rate drops below a predetermined absolute rate for a predetermined time.

10. A baby monitor according to claim 6 adapted to alarm when heart rate drops by a predetermined proportion of a measured baseline within a predetermined time period.

11. A baby monitor substantially as described herein with reference the accompanying drawings.
